(19) United States
(12) Patent Application Publication
(10) Pub. No.: US 2024/0082128 A1
(43) Pub. Date: Mar. 14, 2024

(54) COMPOSITION FOR USE IN DEGRADATION OF BIOFILM OR PREVENTION OF BIOFILM FORMATION

(71) Applicant: Pharmiva AB, Lund (SE)

(72) Inventors: Åke Lindal, Malmö (SE); Anna Runnsjö, Furulund (SE)

(73) Assignee: Pharmiva AB, Lund (SE)

(21) Appl. No.: 18/273,001

(22) PCT Filed: Jan. 27, 2022
(86) PCT No.: PCT/EP2022/051927

§ 371 Date: Jul. 18, 2023

(30) Foreign Application Priority Data
Jan. 27, 2021 (EP) 21153819.4

(51) Int. Cl. A61K 8/37; A61K 8/22; A61Q 17/00

(52) U.S. Cl. CPC A61K 8/375; A61K 8/22; A61Q 17/005; A61K 2800/5922; A61K 2800/805

(57) ABSTRACT

The present invention relates to a composition for use in degradation of biofilm or prevention of biofilm formation in a subject, wherein the composition comprises at least one crystalline aliphatic monoglyceride.

### TECHNICAL FIELD

The present invention relates generally to compositions comprising crystalline lipids for use in degradation of biofilm and for the prevention of biofilm formation. The present invention also describes a method for degrading or preventing biofilms on a skin surface of the human body by applying the composition described herein.

### BACKGROUND ART

The normal flora may be defined as the mixture of microorganisms that live on or in another living organism, such as a human or animal host, without causing disease. In recent years, the role of the normal flora has been subject to many studies in attempt to understand its importance to the host. This has so far led to the realization that the role of the normal flora on the overall wellbeing of a host can be measured from its influence on host anatomy, physiology, susceptibility to pathogens, and morbidity.

The normal flora in humans usually develops in an orderly sequence, or succession, after birth, leading to the stable populations of bacteria that make up the normal adult flora. The main factor determining the composition of the normal flora in a body region is the nature of the local environment, which is determined by multiple factors, including pH, temperature, redox potential, and oxygen, water, and nutrient levels. Other factors which are specific to the given environment of the normal flora may also play roles in flora control, an example being the production and constitution of saliva which may influence the oral and upper respiratory tract flora.

As evident from the above, the normal flora may be influenced by a variety of positive and negative, internal and external factors, some of which include genetic disposition to illnesses, social behaviour, diet, medication, etc. The strength or balance of the normal flora will thus play a crucial factor in its ability to protect the host from these above-mentioned factors and maintain a healthy environment on or in the host.

Every now and then, a host is subjected to enough stress causing the normal flora to change into an imbalanced flora, also known as dysbiosis. An example of such stress may be the subjection of a host to a course of antibiotics, in which the normal flora of the host may also be detrimentally influenced. In such cases, the local flora will be greatly influenced by the microorganisms which were left unharmed by the given antibiotics and these microorganisms may consequently predominate the given local flora for a period. For a healthy individual, most often the normal flora will be re-established after a short period of time, however, in the case of immunocompromised individuals or for people undergoing prolonged antibiotic treatment due to infection caused by multi-resistant microorganisms, the composition of the flora may be permanently changed.

Another consequence of an imbalanced normal flora is the formation of local biofilm populations in the host. Formation of biofilm is one of the major reasons for treatment failures in managing infections, mostly because mature biofilms display an increased antimicrobial tolerance and immune response evasions. Since most drugs penetrate slower through biofilm than in body fluids, the pathogens are protected in the biofilm environment.

Biofilms are formed of aggregates of microorganisms in which cells that are frequently embedded within a self-produced matrix of extracellular polymeric substances (EPSs) adhere to each other and/or to a surface. The formation of biofilm is a means of the microorganism to protect itself from endogenous and exogenous stress. This has been demonstrated by Jang and Kim et al. (Sci. Rep. 2016, 6: 21121, doi:10.1038/srep21121) who could show that when microorganisms were exposed to a small amount of hydrogen peroxide (endogenous stress, 5 nM) biofilm formation was promoted.

The self-produced matrix of EPSs is a polymeric conglomeration generally composed of extracellular biopolymers in various structural forms. These polymers include polysaccharides, glycoproteins and polypeptides. Biofilms may form on living or non-living surfaces and are commonly found in natural, industrial, and hospital settings. Examples of natural settings include on the surface of skin, within body cavities and on implanted devices, which may include, but are not limited to, bone/dental/breast implants, catheters, and other biomedical devices suitable for implantation.

The specific composition of the biofilm may depend on the environment in which the biofilm is formed and the nutrients available. Furthermore, the specific constitution of the biofilm also influences how it reacts in a different manner when exposed to biofilm degrading agents. It is therefore important to find general mechanisms for degradation of biofilm to avoid treatment failures.

The effect of peroxides in general and hydrogen peroxide in particular on biofilms is described in the literature.

If using hydrogen peroxide as an active ingredient in a formulation, it is important to have hydrogen peroxide present at the site of action for some time. The mechanism of degradation of biofilms by hydrogen peroxide can be calculated as reduction of biomass as described in Christensen and Trönnes et al. (Biofouling 1990; 2(2); pp. 165-175). In this investigation, the concentration of hydrogen peroxide needed, as a sole agent to reduce the amount of biofilm by 85% for one hour, was 0.5% w/w.

The use of hydrogen peroxide as an antiseptic agent to treat for example skin infections in humans or animals is limited by the toxicity of the substance, in that even small concentrations of hydrogen peroxide can cause irritation. Depending on the length of exposure and concentration, hydrogen peroxide can give rise to mild itching or even a burning sensation at the site of exposure. Medicinal products, including antiseptics, typically contain 1-5% w/w hydrogen peroxide and domestic products, such as disinfectants, may contain 3-6% w/w hydrogen peroxide. When used at these concentrations, hydrogen peroxides are generally regarded safe, however, may still give rise to local irritation. At the same time, a too low concentration of the hydrogen peroxide may result in a lack of therapeutic effect. This may result in the beneficial microorganisms being adversely affected which may result in the formation of a new microbial population formed by for example multi resistant pathogenic microorganisms.

Other types of compositions with antimicrobial or antibiofilm properties include synergistic compositions comprising two or more agents, which when administered together, result in a better effect of the individual agents. Examples of such synergistic mixtures are reported in WO 2013/169231 A1 and U.S. Pat. Nos. 9,023,891, 9,271,495, 8,834,857, 8,926,997, 8,795,638, 8,734,879, and 8,193,244, which disclose salts comprising a cation N^{α}—C₈-C₁₆-alkanoyl dibasic amino acid (C₁-C₄)-alkyl ester together with various anions selected from the group consisting of halide, nitrite, nitrate, phenolate, polyphenolate, carboxylate, hydroxycarboxylate, hyaluronate, antibiotic anions and amino acids.

Other examples include U.S. Pat. No. 8,604,073 which discloses medical devices incorporated with a biofilm-inhibiting composition comprising Ethyl lauroyl arginate HCl (also known as lauric arginate and LAE) and an antibiotic as well as U.S. Pat. No. 8,604,073 which discloses an antimicrobial composition comprising LAE and one or more antibiotic. WO 2012 013577 discloses an inhibiting effect of LAE on biofilm formation on surgical implants and catheters.

Gil et al. (Antimicrobial Agents and Chemotherapy, July 2017 Vol. 61 Is. 7) reports the use of stainless-steel K-wires coated with monolaurin solubilized in ethanol using a simple, but effective, dip-coating method.

U.S. Pub. Appl. No. 2015/0010715 discloses antimicrobial coatings composed of a hydrogel and a bioactive agent including a substantially water-insoluble antimicrobial metallic material (silver sulfadiazine) that is solubilized within the coating.

U.S. Pat. No. 6,638,978 lists a preservative formulation for food and cosmetics consisting of glyceryl monolaurate (monolaurin or “ML”), a mixture of caprylic and capric acid and propylene glycol in an aqueous base.

U.S. Pat. No. 4,002,775 discloses the discovery that highly effective and yet food-grade microbicides are provided by monoesters of a polyol and a C12 aliphatic carboxylic fatty acid.

WO2016048230A and WO2018215474A1 describes foam-forming formulation and method of treating an infection in a body cavity. The foam-forming formulation may further contain an active ingredient, monoglyceride crystals, at least one acid and/or buffer and a blowing agent.

Common to all the compositions and active agents reported above is their antimicrobial and antibiofilm effect. However, due to the increase in multi-resistant isolates, as well as the worldwide spread thereof, there remains an urgent need for safe and efficient antimicrobial and antibiofilm agents as alternatives to antibiotics.

In view of the above, there exists a need for new, safe and efficient treatment regimens for establishing and maintaining a systemic-wide normal flora of a host. In particular, there remains a need for chemically stable, antibiotic-free compositions for use in degradation of biofilm and prevention of biofilm formation.

### SUMMARY OF THE INVENTION

In view of the above, it is therefore an object of the present inventive concept to provide an effective and safe composition for use in the degradation of biofilm or prevention of the formation of biofilm in a subject.

Accordingly, a first aspect of the present invention relates to a composition for use in degradation of biofilm or prevention of biofilm formation in a subject, wherein the composition comprises at least one crystalline aliphatic monoglyceride.

The composition may be a pharmaceutical composition. The composition may further comprise a buffering agent, a salt and/or water. The buffering agent may be phosphate buffer, lactate buffer or other weak acids/bases suitable for human use.

The inventors have surprisingly found that use of a composition comprising at least one crystalline aliphatic monoglyceride is efficient in both degrading biofilm in a subject, see FIGS. 1 and 2. Furthermore, the use of the composition comprising at least one crystalline aliphatic monoglyceride has also been shown to be efficient in preventing the formation of biofilm, see FIG. 3. Without the wish to be bound by any particular theory, it is believed that at least one crystalline aliphatic monoglycerides, such as for example monolaurin or monomyristine, is efficient in degrading the biofilm and preventing the formation of same. The effect is observed both for compositions with one crystalline aliphatic monoglyceride, e.g. monolaurin, and for compositions with a combination of crystalline aliphatic monoglyceride, see FIG. 2. Without the wish to be bound by any particular theory, it is believed that the crystallinity of the monoglyceride(s) of the composition is essential for the degradation of the biofilm. This is supported by FIG. 2 which shows that crystalline laurate and crystalline myristate are both more efficient in degrading biofilm polymers than amorphous, non-crystalline laurate. It is thus particularly surprising that the crystalline monoglycerides of the present invention are not only involved in providing a stable composition for use in body cavities or on skin areas, but also are efficient in degrading biofilm polymers.

The inventors have further found that the use of a composition comprising at least one crystalline aliphatic monoglyceride is efficient in decreasing biofilm biomass and biomass viability, see FIGS. 3 and 4.

While the antimicrobial activity of monoglycerides have previously been described, see, for example, Strandberg et al., Antimicrob. agents and Chemother., Vol. 54, No. 2: 597-601 (2010), it is surprising that the crystalline aliphatic monoglycerides of the present invention is also efficient in degrading and preventing the formation of biofilm. To the inventors' knowledge, such dual function of the monoglycerides have not been described previously, and thus the present invention offers a new therapeutic application. Strandberg et al. (2010) further reports that while the monoglyceride glycerol monolaurate has an inhibitory effect on Candida and Gardnerella vaginalis, the compound does not influence Lactobacillus count when the compound is used for treatment of bacterial vaginosis. In that sense, the use of monoglycerides in the composition of the present invention, has the further advantage of not influencing the commensal, beneficial microorganisms of the host, thus, providing ideal circumstances for maintaining a healthy normal flora in the host.

The composition may further comprise an active agent such as a peroxide. Thus, in one embodiment, there is provided a composition for use in degradation of biofilm or prevention of biofilm formation in a subject, wherein the composition comprises at least one crystalline aliphatic monoglyceride and a peroxide.

The inventors have also found that the combination of at least one crystalline monoglyceride and a peroxide compound in a composition is efficient in both degrading biofilm in a subject and preventing the formation of same. Although it is known that peroxides may decrease the mass of biofilm, the efficacy of the combination of peroxide and crystalline monoglycerides according to the invention resulted in a surprisingly high efficacy.

Without the wish to be bound by theory it is believed that some interaction between peroxides, a non-limiting example being hydrogen peroxide, and crystalline lipids, such as for example monolaurin and monomyristine, is responsible for the observed synergistic effect against biofilm. Furthermore, it has been found that the compositions of the present invention not only provide an antibiofilm effect through an antimicrobial effect, i.e. killing only the microorganisms contained in the biofilm, but that the agents of the mixture disrupt the biofilm itself by influencing the biopolymers, i.e. the matrix of the biofilm. This alternative target of the composition provides a surprisingly efficient degradation of the biofilm in that the disruption of the matrix protecting the organisms living within the biofilm, results in the exposure of the microbes to the active agents without the need for high concentration of these. The alternative target of the composition also provides a surprisingly efficient prevention of the formation of biofilm, both by breaking down the biofilm matrix and by preventing the formation thereof.

The inventors have surprisingly found a 256 to 512 times higher antimicrobial and antibiofilm effect of the lipid mixtures of the present invention compared to using hydrogen peroxide alone as active. This effect was seen from comparison of the rate of biofilm degradation and duration of the effect of the tested mixtures compared to reference. The high efficacy of the invented composition is an advantage over the currently available formulations suitable for treatment of biofilm. The high effect makes it possible to use a lower amount of peroxides and/or other active agents while still maintaining the effect of the composition, thus making the product suitable for general use due to low toxicity and low irritation. The inventors have further shown the surprising possibility in a very simple manner of providing the degradation and prevention of biofilm formation in a subject. The simplicity of composition further improves the characteristics of the composition, including little to no toxicity and little to no irritation when used. This also allows for the use of the composition as a prophylactic agent without causing undesired irritation to the subject. The prophylactic use of the composition of the present invention ensures the prevention of biofilm formation in natural, i.e. body cavity, wounds and skin surfaces of a subject.

In the context of the present invention, biofilm formation may be the result of an infection with a pathogenic microorganism. The pathogenic microorganism will induce the formation of biofilm to evade the host defence systems of the subject. However, since most microorganisms, including both pathogenic and non-pathogenic microorganisms, are capable of producing biofilms it is also possible that the biofilm is formed by a non-pathogenic microorganisms. Biofilms may harbour mixtures of pathogenic and non-pathogenic microorganisms. By staying dormant and hidden from the immune system, the microorganisms in the biofilm may cause local tissue damage and later cause an acute infection. Since the composition of the present invention is capable of degrading the biofilm, irrespective of the microorganisms hosted therein, the composition is particularly beneficial in the treatment of biofilm-associated diseases and conditions involving the formation of biofilm, but not necessarily involving an active disease progression and symptoms of disease. In other words, the composition of the present invention offers an indirect treatment of pathogenic microorganisms based on the ability of the composition in degrading biofilm. In that sense, the composition of the present invention distinguishes itself from the conventional antimicrobial treatment regimes, by targeting a different aspect of an infection, namely the biofilm. The composition of the present invention is thus particularly useful in treating biofilm-associated conditions.

The present invention further provides the use of a composition comprising at least one crystalline aliphatic monoglyceride for degradation of biofilm or prevention of biofilm formation in a subject. In the context of the present invention, the use of the composition for the degradation of biofilm or prevention of biofilm formation in a subject comprises applying the composition to a skin surface, e.g. as a cosmetic treatment. Such cosmetic treatment may be to improve or maintain the normal flora of a skin area in order to for example improve or avoid foul smell which may be caused by biofilm formation on the skin surface.

In an embodiment of the present invention, the concentration of the peroxide in the composition is less than 0.9% w/w. A benefit of the low concentration of peroxide in the composition is that the beneficial bacteria present, whether it is lactobacilli, bifidus or any other species, may have different sensitivity towards peroxides and thus respond differently to the composition.

A major issue when treating diseases in which biofilm formation is part of the infection, is the antibiotic resistance of the population contained within the biofilm. Thus, courses of antibiotics often fail to sufficiently degrade and eradicate biofilm. Antiseptics with less specific action than antibiotics include peroxides, halogens, such as chlorine and iodine, phenols and alcohols, as well as phenolic and nitrogen compounds. However, the lower specificity of antiseptics leads in general to a larger risk of toxicity. Thus, most antiseptics are unsuitable for administration into body cavities. One that is suitable is hydrogen peroxide (HP) or H₂O₂. Accordingly, in one embodiment of the present invention, the peroxide compound of the composition is hydrogen peroxide or benzoyl peroxide.

It is known that peroxides and in particular hydrogen peroxide is an effective antiseptic compound and that most microorganisms are sensitive to HP. The inventors of the present invention have found that the combination of crystalline aliphatic monoglyceride and peroxide is capable of eradicating the relevant bacteria when present in an effective amount. Hydrogen peroxide has been administered to humans for over 100 years and one problem that has limited the use of HP has been the auto-oxidation of hydrogen peroxide. This phenomenon leads to a rapid degradation of HP as soon as HP is exposed to reactive matter. The fast reaction leads to boiling and development of oxygen, a degradation product of HP, whereby the HP is consumed within minutes or seconds. It has been found, that with the presence of crystalline monoglycerides derived from aliphatic carboxylic fatty acid, of preferably from C10 to C16 carbon chain length, the rate of degradation of HP at the site of action can be regulated and optimized for maximum effect. This procedure has been described in the literature for use on skin at higher concentrations of HP. This procedure has however not been demonstrated for use in body cavities or for lower concentrations of HP, such as 0.5% and below.

In an embodiment of the present invention, the at least one monoglyceride of the composition is an aliphatic carboxylic fatty acid glyceride of C10 to C16, such as C10, C11, C12, C13, C14, C15 or C16 in length or any combinations thereof. In a preferred embodiment, the at least one monoglyceride is C12 to C14, or a combination thereof.

In an embodiment of the invention, the at least one monoglyceride is crystalline.

In one embodiment, the at least one monoglyceride is selected from glycerol monocaprate, glycerol monolaurate, glycerol monomyristate and glycerol monopalmitate.

In one embodiment, the at least one crystalline monoglyceride of the composition is monolaurin, monomyristine or a combination of both. Monolaurin, also known as glycerol monolaurate (GML), 1-glycerylmonolaurate, glyceryl laurate and 1-lauroyl-glycerol, is a C12-monoglyceride and is the monoester formed from glycerol and lauric acid. Monomyristine, also known as glyceryl 2-myristate and 1-glycerylmonomyristate, is a C14-monoglyceride. When using more than one monoglyceride in the composition, the amount of and the ratio between the monoglycerides can be varied depending on the required viscosity of the final product.

In an embodiment, the at least one crystalline monoglyceride of the composition is monolaurin and monomyristine and the ratio between monolaurin and monomyristine is from 1 to 10 to 10 to 1. The inventors have surprisingly found that when using two monoglycerides, such as monolaurin and monomyristine, the effect of HP on biofilms is enhanced vis-à-vis the effect of using a single monoglyceride. The combination of monomyristine and monolaurin decreases the melting point of the crystals of the lipids. Pure crystals of monolaurin and monomyristine melt at 39° C. and 41° C., respectively, whereas all mixtures of monolaurin and monomyristine in the range from 1 to 10 to 10 to 1 melt at about 33° C. This phenomenon is also known as a eutectic system and is the result of the two substances being soluble in each other and being able to form a homogenous mixture. Without the wish to be bound by theory, it is believed that the decrease in the lowest possible melting temperature of the two monoglycerides results in a faster and more efficient release of the substances within the composition including the monoglyceride mixture. It is thus believed that by decreasing the lowest possible melting temperature of the two monoglycerides, an improved release of the active substance, i.e. peroxide, is achieved at the site of application of the composition. Without wishing to being bound by theory, it is further believed that the homogenous mixture of the monoglycerides results in a better distribution of the composition at the site of application and thus an improved therapeutic efficacy. In the treatment of biofilm-associated infections, distribution of the active substance is an important factor for ensuring that the entire infection-causing population is affected by the composition, i.e. that the entire biofilm is affected by the composition, such that micro-populations evading treatment, is not formed. Without the wish to be bound by theory, it is believed that the combination of peroxide and monoglycerides results in a synergistic effect of the mixture. The unique combination of monoglycerides results in an improved disruption of the polymers of the biofilm which in turn results in a lower amount of peroxide needed for causing the antibiofilm (antiseptic) effect. The composition may also be ideal for the preservation of beneficial bacteria of the natural flora of humans and animals by its selective effect on microorganisms considered harmful for the host.

In one embodiment of the invention the composition consists of at least one crystalline aliphatic monoglyceride and peroxide. In a further embodiment the composition consists of a combination of monolaurin and monomyristine. The ratio between monolaurin and monomyristine may be from 1 to 10 to 10 to 1.

The monoglyceride of the composition should be at least partly in its crystalline state, more preferable to 50% and even more preferable to 70% and most preferably to 80% determined by differential scanning calorimetry.

Crystalline lipids are defined by a continuous repeated structure in three dimensions, but the nature of the repetition may not be the same in all directions. The crystals may contain bilayers of water and lipid creating a repeated structure of water and lipid layers in one direction and lipid crystals in two directions. One way to detect crystallinity is to study birefringence in microscope. For example, a definition of a lipid lamellar crystal is a solid crystal with three-dimensional continuity having the same repeated cells in two dimensions, but a different one in the third dimension (from Small, The lipid handbook), which can be established by wide angle X-ray ref. The crystallinity of monoglycerides in the compositions can be determined by differential scanning calorimetry (DSC).

There are several advantages associated with the use of (solid) lipid crystals. Since the crystalline state in general is the lowest energetic state very little will happen with the structure during storage. Such stable constituents are regarded as a great advantage in the development of pharmaceutical products.

In the context of the present invention, in an embodiment comprising both monoglyceride and peroxide, the composition may be prepared by the following method; melting the least one monoglyceride together with water and optionally a suitable buffer or acid at 75° C. for 15 minutes to form a monoglyceride composition; cooling the monoglyceride formulation in a cooling process to reduce the temperature of the formulation 1° C. to 5° C. per minute; stopping the cooling process when the monoglyceride composition is about 35° C. and allow the at least one monoglyceride to crystallize without further cooling; and allowing the formulation to reach ambient temperature. During the cooling, the monoglyceride crystalizes from alpha to beta prime crystals, which generates heat through the process known as exotherm crystallization. The peroxide of the composition can be included at any time during the manufacturing of the composition, however, in particular if the peroxide compound is HP, the peroxide compound is preferably added during the melting or before the composition reaches ambient temperature, since the viscosity is very high at ambient temperature. The above-mentioned method of preparing the composition of the present invention can also be carried out in the same manner in its broadest sense without the addition of peroxide to the composition.

An aspect of the present invention relates to a composition consisting or essentially consisting of a crystalline aliphatic monoglyceride, one or more salts, a buffer system and water.

Another aspect of the present invention relates to a composition consisting or essentially consisting of a crystalline aliphatic monoglyceride, a peroxide, one or more salts, a buffer system and water. In the context of the present invention, a buffer system should be understood in its normal sense, and may be formed from a single species or a mixture of a weak acid and its conjugate base, or a weak base and its conjugate acid. A strong acid or base is used to adjust to the desired pH if necessary in the usual manner.

Said composition may consist of a combination of monolaurin and monomyristine. The ratio between monolaurin and monomyristine of said composition may be from 1 to 10 to 10 to 1.

Without the wish to be bound by any particular theory, it is believed that the one or more salts of the composition acts as a further antimicrobial agent in that it causes a general stressing of the microorganisms treated with the composition. Furthermore, salts may be used to obtain a desired tonicity. In some embodiments the tonicity of the composition is isotonic with the site of application.

Suitable salts which may be used in the composition according to the present invention, includes, but are not limited to one or more of sodium ethylenediaminetetraacetic acid (EDTA), sodium pyrophosphate, sodium stannate, and sodium oxalate. Thus, in one embodiment, the one or more salts used in the composition according to the invention is one or more salts selected from the group comprising sodium EDTA, sodium pyrophosphate, sodium stannate, sodium oxalate, as well as any combination thereof. In one embodiment, the one or more salts used in the composition according to the invention is sodium pyrophosphate, sodium stannate, and sodium oxalate.

In an embodiment of the present invention, the composition is a composition provided as a mousse, tampons, creams, gels, vaginal suppositories and vaginal tablets. This is achieved by using the composition directly in cream or gel formulations, by mixing the composition with air to form a mousse product or by freeze- or spray-drying to form a dry formulation, which may for example be applied to tampons or in tablets. Independent of the use of the composition, the final product is effective in reducing biofilm in infected body cavities or areas of the skin. Suitable total amounts of monoglycerides for the purpose of making a mousse is from 10% to 30%, more preferably from 15% to 25%, based on the final composition. For the preparation of a cream or a gel, the suitable total amounts of monoglycerides are from 5% to 30%, more preferably from 10% to 27%, based on the final composition. For low viscosity gels and sprays, a suitable total amount of crystalline monoglycerides is from 0.1% to 10% based on the final composition. Furthermore, in an embodiment of the present invention, the composition further comprises a non-lipophilic propellant when provided as a mousse. In one embodiment of the present invention, the non-lipophilic propellant is air or a gaseous mixture simulating one of air, oxygen, nitrogen, and carbon dioxide. In another embodiment, the non-lipophilic propellant is air or a gaseous mixture simulating air, or other combinations of oxygen, nitrogen and carbon dioxide. In one embodiment, the non-lipophilic propellant is air. In one embodiment, the non-lipophilic propellant is air or gaseous mixture simulating air. Furthermore, by administering the product in the form of a foam, the entire volume of the cavity or the entire surface of the area can be filled. The foam is constructed to physically decompose, i.e. to melt, at skin temperature and thereby the entire surface of the cavity will be treated.

In another embodiment of the present invention, the composition further comprises a solubilizing agent. The solubilizing agent can make up the balance of the composition, and the resulting composition may thus be more stable and homogenous. In one embodiment, the solubilizing agent is selected from polar alcohols or esters thereof accepted for use on skin or in body cavities, exemplified but not limited to polyethylene glycol, glycerol, propylene glycol and ethanol.

In one embodiment of the present invention, the composition is administered to an infected body cavity or areas of the skin. In the context of the present invention, an infected body cavity or areas of the skin of a subject is comprising a mixture of biofilm and dead and alive microorganisms. The microorganisms may be dormant, i.e. alive but having entered a hardy, non-replicating state. The microorganisms are generally pathogenic to the host. Alternatively, when the composition is administered to a body cavity or areas of the skin for the prevention of biofilm formation in a subject, the body cavity or areas of the skin may or may not comprise biofilm and dead and alive pathogenic microorganisms. For the use of the composition for prevention of biofilm formation, the composition may be applied to an area in which biofilm is prone to occur, for example, but not limited to, an area of the skin or body cavity undergoing a treatment which will affect the normal flora of the area of the skin or body cavity.

In an embodiment, the composition is administered to an infected body cavity or areas of the skin, which is caused by an infection with a pathogenic microorganism. In one embodiment, the infection is caused by Gardnerella vaginalis, Candida albicans or a combination of both. G. vaginalis is a facultatively anaerobic Gram-variable bacteria which is involved, together with many other bacteria, mostly anaerobic, in bacterial vaginosis in women as a result of a disruption in the normal vaginal microflora. C. albicans is an opportunistic pathogenic yeast which is a common member of the human gut flora. C. albicans can also survive outside the human body and is also frequently detected in the gastrointestinal tract and mouth of healthy subjects. In the context of the present invention, C. albicans is an important microorganism to study since it is the most common fungal species isolated from biofilms either formed on implanted medical devices or on human tissue. In addition, hospital-acquired infections by C. albicans have become a cause of major health concerns.

Infection of the body cavity or areas of the skin may be caused by a single genus or species of microorganisms or combinations of more than one genus or species of microorganisms. Biofilms may host a diverse range of microorganisms. Consequently, an infection of the body cavity of skin area may be the result of a mixture of pathogenic and commensal microorganisms which are clustered in the same biofilm matrix.

In one embodiment, the infection is caused by a lack of commensal microorganisms in the infected body cavity of areas of the skin. Commensal bacteria are beneficial bacteria which inhabits mucosal and epidermal surfaces in humans and plays an important role in defence against pathogens. In such cases, the infection occurs as a result of the disruption of the normal flora of the host, thus, leading to conditions where pathogens may evade and spread to form biofilm and/or infection. In the context of the present invention, the commensal microorganisms found in body cavities may be species of Lactobacillus, Streptococcus, Bifidobacterium, and Actinomyces, and mixtures thereof. The commensal microorganisms found on areas of the skin may be Propionibacterium species, Staphylococcus, Corynebacterium species, Malassezia species, and mixtures thereof.

In another embodiment of the present invention, the pH of the composition is selected in accordance with the pH of the healthy tissue at the site of application tissue and/or mucous membrane at the site of application. The pH of the product can be selected according to the intended environment. The pH of the product can also be selected according to the subject in need of the treatment. For example, for vaginal applications a pH of 3.5 to 5 of the composition is suitable. For example, for topical applications a pH of 4 to 6 of the composition is suitable. In one embodiment, the pH of the composition is in range of pH 3.5 to 6. In another embodiment, the pH of the composition is in range of pH 4 to 6. The pH of the composition may be maintained using a suitable buffer for the desired pH range, such as a lactic acid and other alfa hydroxy acid buffer systems. In the context of the present invention, a suitable buffer may be any physiologically acceptable buffer effective in the pH range of pH 4 to pH 6. Thus, in an embodiment, the pH of the composition is maintained with any physiologically acceptable buffer effective in the pH range of pH 4 to pH 6. In one embodiment, the lactate/lactic acid is added to the composition as a buffer.

In another embodiment, the lactic acid is the d-isomer of lactic acid. Without wishing to be bound by any particular theory, it is believed that the addition of a buffer to the composition further promotes the antimicrobial and antibiofilm effect of the composition in that the buffer ensures a pH which promotes the growth of beneficial commensal bacteria, such as lactobacilli.

For application of the composition in wounds, the composition may be maintained at a neutral pH using a physiologically acceptable buffer, such as a phosphate buffer or other buffer systems suitable for human use. For applications in infections on skin the composition should have pH at 5.5 or lower. A nonlimiting example of a suitable buffer is a lactate buffer. The person skilled in the art will know how to adjust the pH of the composition according to the intended environment. In one embodiment, the pH of the composition is adjusted using sodium hydroxide and maintained at the pH using a physiologically acceptable buffer, such as a lactate buffer. Most suitably the composition should have a pH at or near the pH of the site of application. The skilled person will know how to determine such pH.

The compositions described herein may be used in combination with any further suitable medically active ingredient such as a drug, a medicament, or an active ingredient. Medically active agents are agents effective in the treatment of skin infections and inflammation, such as in the treatment of conditions in wounds and in body cavities. Non-limiting examples of medically active agents are anti-inflammatory agents, antibiotics, antivirals, antifungals, antipsoriatic agents, agents for the control of humidity or pH in skin as well as agents for the treatment of acne.

The present invention further provides a method for degrading biofilms on a skin surface of the human body, comprising applying a composition comprising at least one crystalline aliphatic monoglyceride to the surface in such a manner that the composition contacts the skin surface. The present invention also provides a method for degrading biofilms on a skin surface of the human body, comprising applying a composition comprising at least one crystalline aliphatic monoglyceride and a peroxide to the surface in such a manner that the composition contacts the skin surface. In one embodiment, the method for degrading biofilms on a skin surface of the human body is a non-therapeutical method. The composition may be formulated as a topical and intra-cavital formulation. The composition can be administered immediately upon the discovery of an infection without any risk of causing antibiotic resistance to the infecting agent and with a high probability of efficient treatment irrespective of the nature of the infecting agent, e.g. bacteria, virus, fungi and flagellates. Also, in order to exercise a medical effect the formulation must be in physical contact with the entire affected tissue. This is achieved with the composition of the present invention.

The present invention further provides a method for preventing the formation of biofilms on a skin surface of the human body, comprising applying a composition comprising at least one crystalline aliphatic monoglyceride to the skin surface in such a manner that the composition contacts the skin surface. Alternatively, the present invention further provides a method for preventing the formation of biofilms on a skin surface of the human body, comprising applying a composition comprising at least one crystalline aliphatic monoglyceride and a peroxide to the skin surface in such a manner that the composition contacts the skin surface.

In one embodiment, the method for preventing the formation of biofilms on a skin surface of the human body is a non-therapeutical method.

A surface temperature of at least 33° C. and ideally around 37° C.-40° C. is considered optimal to obtain effective release of active ingredients while still preserving the structure and activity of the active substances. Suitable catalysts may be added to the composition at application to the infected area. If such catalysts are added to the composition surface temperatures could be less than 33° C. Such catalysts include Fe, Mg, Mn and Cu which are suitable for compositions applied to inanimate surfaces.

Other aspects and advantageous features of the present invention are described in detail in relation to the compositions and illustrated by non-limiting working examples below.

### BRIEF DESCRIPTION OF THE FIGURES

The above, as well as additional objects, features, and advantages of the present invention is better understood through the following illustrative and non-limiting detailed description of embodiments of the present invention, with reference to the appended drawings, wherein:

FIG. 1 shows biofilm extracellular matrix proxi degradation after incubation with compositions of the inventions,

FIG. 2 shows biofilm extracellular matrix proxi degradation after incubation with additional compositions of the inventions,

FIG. 3 shows biofilm biomass of C. albicans and G. vaginalis after incubation with compositions of the inventions, and

FIG. 4 shows biofilm viability of C. albicans and G. vaginalis after incubation with compositions of the inventions.

### DETAILED DESCRIPTION

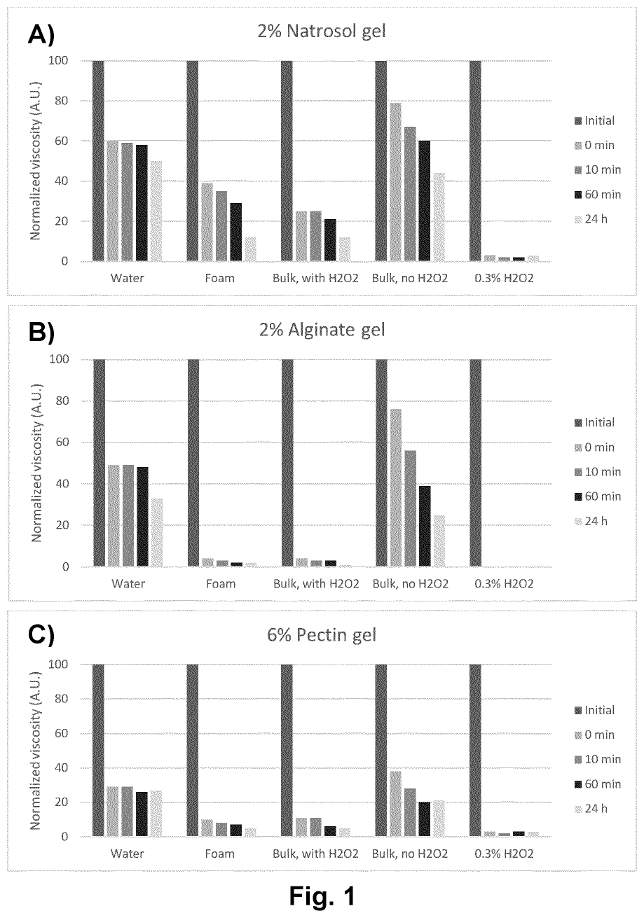

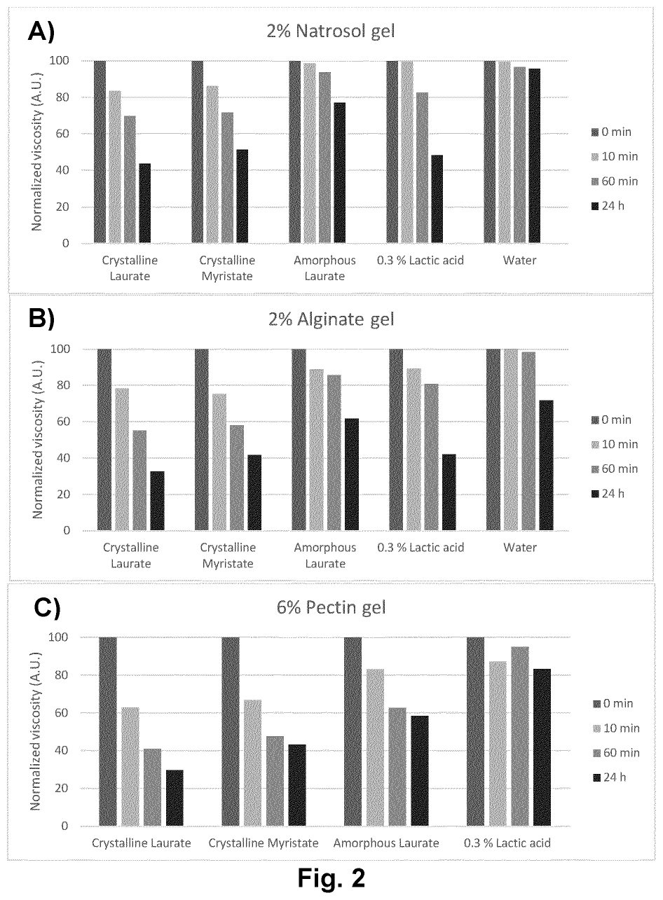

FIGS. 1 and 2 show the results obtained from the experiments described in Example 2. In particular, FIGS. 1 and 2 show the effect of crystalline monoglycerides on polymer films formed by gels of Natrosol (FIGS. 1A and 2A), Alginate (FIGS. 1B and 2B) and Pectin (FIGS. 1C and 2C), which are used as models for extracellular polymer substances (EPS) in biofilms. Viscosity measurements on these model gels were done before and after addition of formulations containing either crystalline or amorphous monoglycerides. The viscosities were normalised to the viscosity of the pure gel (viscosity value measured before addition of any formulation). As seen in FIG. 2, it was shown that crystalline monolaurate and crystalline myristate efficiently degrades the model gels. It was found that amorphous laurate did not degrade the polymers to the same extend as the crystalline monoglycerides. A slight degradation of two of the model gels, Natrosol gel and Alginate gel, could also be seen after addition of 0.3% lactic acid solution.

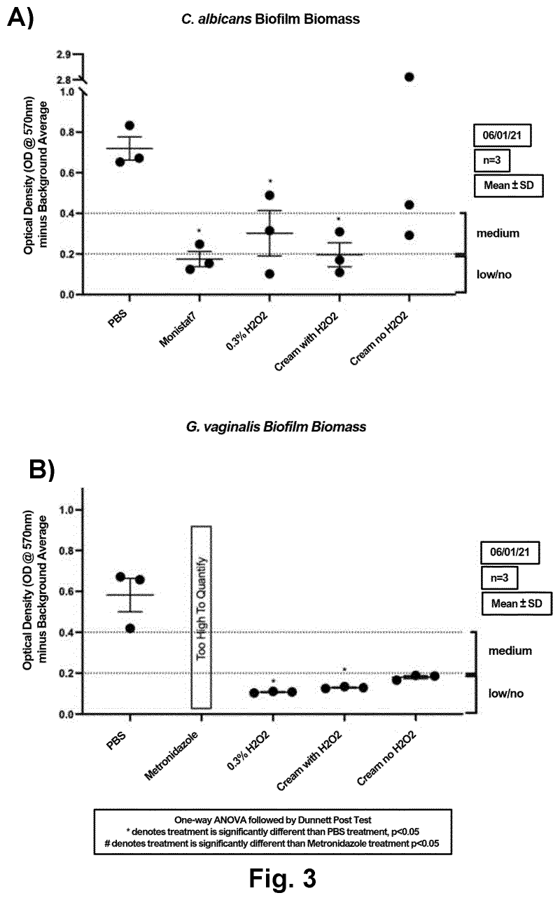

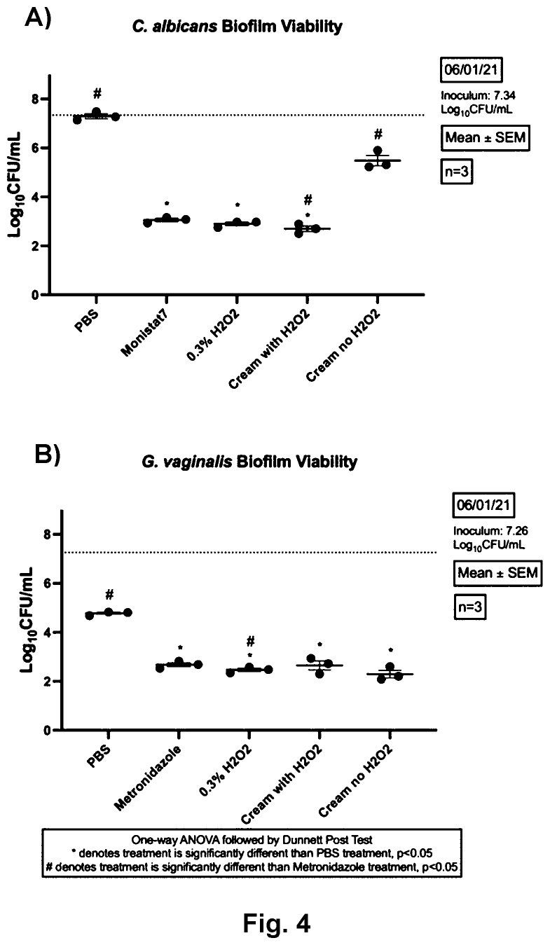

FIGS. 3 and 4 show the results obtained from the experiments described in Example 3. In particular, FIG. 3 shows that the bulk cream, 0.3% H₂O₂ solution and bulk cream without H₂O₂ can all efficiently remove biomass from biofilms formed by C. albicans (FIG. 3A) and G. vaginalis (FIG. 3B) after only 24 hours incubation. It was also found that the bulk cream was capable of decreasing the biofilm biomass (as shown by the low optical density, OD=570 nm, values reported in FIG. 3) of C. albicans with an efficiency comparable to that of the clinical comparator Monistat-7, which is an antifungal medication used to treat vaginal yeast infections. As seen in FIG. 4, bulk cream, 0.3% H₂O₂ solution and bulk cream without H₂O₂ all efficiently decrease the viability of G. vaginalis (FIG. 4B). It is seen that bulk cream without H₂O₂ results in a lower Log₁₀CFU/mL value (more efficient decrease in viability of cells) than that of the clinical comparator Metronidazole, which is an antibiotic that commonly is used to treat bacterial infections, including, but not limited to, bacterial vaginosis.

Generally, all terms used herein are to be interpreted according to their ordinary meaning in the technical field, and applicable to all aspects and embodiments of the invention, unless explicitly defined or stated otherwise. All references to “a/an/the [composition, lipid, peroxide, propellant, agent, etc.]” are to be interpreted openly as referring to at least one instance of said composition, lipid, peroxide, propellant, agent etc., unless explicitly stated otherwise.

In the context of the present invention degradation of biofilm is to be understood as any destruction of the biofilm formation or degradation of the components or polymers of the biofilm. Viscosity may be measured to determine length of the polymers and thereby the level of degradation of the biofilm, i.e. the shorter the polymer, the lower the viscosity, the more degraded the biofilm is. Thus, lowered viscosity of the polymer mixtures may be seen as the result of polymers breaking apart, which in turn provides indications that the given composition is effective in breaking up biofilm. A Brookfield viscometer may be used to measure the viscosity.

In the context of the present invention, the term “composition” is used interchangeably with “pharmaceutical composition”, “formulation” and “pharmaceutical formulation” and describes a mixture suitable for use in degradation of biofilm or prevention of biofilm formation in a subject. In the context of the present invention, the term “subject” refers to healthy individuals as well as individuals which are suffering from infections involving biofilm. It is to be understood that an individual with infections involving biofilm may be symptom-free.

In the context of the present invention, when a bulk cream is used for the foam product it can be packed in spray containers consisting of a metal can and an aluminium/polymer laminate internal bag. For the foam product, a propellant is added to the bulk cream. The same propellant can be used inside and outside the laminate bag. Air is used as the preferred propellant when the composition is to be applied to a human or animal since air is a physiologically safe ingredient.

In the context of the present invention, the term “crystalline” used to describe monoglycerides and lipids refers to the crystal form of the compound, i.e. crystal form of monoglyceride and crystal form of lipids. To obtain the desired crystallisation, the monoglycerides used in the composition must be at least 90% pure.

Monoglycerides usable according to the invention can be any available commercial product.

Furthermore, the propellant maintains the crystalline structure of the monoglycerides both in the container during shelf life and after having produced a foam.

In the context of the present invention, the term “healthy tissue” refers to tissue of a living creature, i.e. a human or animal, which is not infected or otherwise imbalanced. The healthy tissue may be on the surface of or within the body of the living creature.

In the context of the present invention, the term “site of application” means the area of the body, externally or internally, which is to subjected to the composition of the present invention. The composition is designed to contact and cover the entire skin surface or tissue where treatment is required. The application may be on the surface of skin, including skin wounds, and in body cavities. Furthermore, the composition is designed so that the contacting form is resistant to removal by flow of wound fluids or other bodily fluids.

Body cavity includes both natural cavities in contact with the surroundings such as vagina, the mouth and throat, the nasal region, the ear, urethra and rectum, and artificial body cavities such as cavities formed during surgical interventions, dialysis, introduction of prostheses or wounds, etc.

### Examples

### Example 1. Manufacture of Compositions of the Invention and Polymer Gels Used as Biofilm Extracellular Matrix Proxi

Compositions

The different formulations and compositions used in the studies on biofilm degradation are listed below. Pure water was added in the studies as reference.
Bulk cream (containing H₂O₂)
Bulk cream (without H₂O₂)
Cream with crystalline monolaurate (no H₂O₂)
Cream with crystalline myristate (no H₂O₂)
Cream with amorphous monolaurate (no H₂O₂)
0.3% lactic acid solution with 0.7% NaOH
0.3% hydrogen peroxide solution
Water

Composition of Bulk Cream

TABLE 1

| Ingredient | g/100 g |
|---|---|
| 1-Glycerylmonolaurate | 4.5 |
| 1-Glycerylmonomyristate | 13.5 |
| Sodium EDTA | 0.05 |
| Sodium pyrophosphate | 0.025 |
| Sodium stannate | 0.04 |
| Sodium oxalate | 0.14 |
| Hydrogen peroxide | 0.306 |
| Lactic acid | 3 |
| 10M NaOH | To pH 3.5 |
| Water to | 100 |

The bulk cream was stored at room temperature until foam packing was undertaken. It was found that the bulk cream had a good shelf life and was able to maintain the crystalline structure of the monoglyceride of the composition sufficiently. Additionally, the original pH as well as the active ingredients were not affected by storage.

Manufacture: EDTA and the sodium salts were dissolved in 75% of the water. Lactic acid and sodium hydroxide were added, and pH adjusted to pH 3.5. Following this, the monoglycerides were added and the mixture was heated to 70° C. to 75° C. and kept at this temperature for 15 minutes while stirring. After 15 minutes, a slow cooling process, i.e. by decreasing the temperature of the mixture with less than 5° C. per minute, was applied until the mixture was about 35° C. At 35° C., crystallization of the monoglycerides begins to occur in the mixture at which point an increase in temperature was observed. After the crystallization was completed, hydrogen peroxide and the remaining water were added to the mixture and the bulk cream was allowed to cool to ambient temperature. The product is either to be used as a cream or stored as an intermediary product awaiting packaging of a foam product.

When the bulk cream is used for the foam product it can be packed in spray containers consisting of a metal can and an aluminium/polymer laminate internal bag. For the foam product, a propellant is added to the bulk cream. The same propellant can be used inside and outside the laminate bag. When air is used as propellant a specific volume is filled up to a predetermined pressure. Air was used as the preferred propellant when the composition was to be applied to a human or animal since air is a physiologically safe ingredient.

Composition of Bulk Cream Containing Crystalline Monolaurate without Hydrogen Peroxide

A cream containing crystalline monolaurin was manufactured according to the manufacture method for bulk cream (as described above) with the exception that the formulation was altered according to Table 2.

TABLE 2

| Ingredient | g/100 g |
|---|---|
| Glycerol monolaurate | 18.0 |
| EDTA | 0.05 |
| Sodium pyrophosphate | 0.025 |
| Sodium stannate | 0.04 |
| Sodium oxalate | 0.14 |
| Lactic acid 90% | 3 |
| Sodium hydroxide | 0.7 |
| Water to | 100 |

Composition of Bulk Cream Containing Crystalline Myristate without Hydrogen Peroxide

A cream containing crystalline myristate was manufactured according to the manufacture method for bulk cream (as described above) with the exception that the formulation was altered according to Table 3.

TABLE 3

| Ingredient | g/100 g |
|---|---|
| Glycerol myristate | 18.0 |
| EDTA | 0.05 |
| Sodium pyrophosphate | 0.025 |
| Sodium stannate | 0.04 |
| Sodium oxalate | 0.14 |
| Lactic acid 90% | 3 |
| Sodium hydroxide | 0.7 |
| Water to | 100 |

Composition of Bulk Cream Containing Amorphous Monolaurate without Hydrogen Peroxide

Preparation of amorphous monolaurate: Amorphous monolaurate were prepared by dissolving monolaurate in ethanol at 50° C. Thereafter ethanol was allowed to evaporate at room temperature.

Manufacture of composition with amorphous monolaurate: The amorphous monolaurate were diluted into a composition with a concentration of 18% monolaurate. The composition was the same as for the cream containing crystalline monolaurate, see Table 2.

0.3% Lactic Acid Solution with 0.7% NaOH

A solution containing 3% lactic acid and 0.7% sodium hydroxide (NaOH) in water was manufactured by dissolving the two ingredients in water.

Manufacture of Polymer Gels for the Study of Biofilm Degradation

Three different gels were used in the study reported in Example 2 below: 2% natrosol gel, 2% alginate gel and 6% pectin gel. FeSO₄ (10 mM) was added to the gels during manufacturing and acts as a catalysator and by mimic oxidation potential in biological fluids. A list of the gels is provided in Table 4.

TABLE 4

| Component | Alginate gel | Pectin gel | Natrosol gel |
|---|---|---|---|
| Sodium alginate | 2 | — | — |
| Pectin | — | 10 | — |
| Natrosol 250 HX | — | — | 2 |
| FeSO₄ | 0.278 | 0.278 | 0.278 |
| Water | 97.722 | 89.722 | 97.722 |
| Total | 100 | 100 | 100 |

The alginate and Natrosol gel were manufactured by adding sodium Alginate/Natrosol to the water solution containing FeSO₄, during stirring. The pectin gel was manufactured by heating the water to 80° C. and add pectin as well as FeSO₄ during stirring. After manufacture, 6 glass vials (60 mL) were filled with 40 mL of the gel.

### Example 2. Effect of Bulk Cream and its Components on Biofilm Extracellular Matrix Proxi Degradation

The formulations and compositions used to test the effect of bulk cream and its components on biofilm extracellular matrix proxi degradation were (See Example 1 for their manufacture):
Bulk cream (containing H₂O₂)
Bulk cream (without H₂O₂)
Cream with crystalline monolaurate (no H₂O₂)
Cream with crystalline myristate (no H₂O₂)
Cream with amorphous monolaurate (no H₂O₂)
0.3% lactic acid solution with 0.7% NaOH
0.3% hydrogen peroxide solution
Water

Study Design

In general, biofilms consist of polymers consisting of polysaccharides, most commonly alginates, extracellular proteins, DNA and small amounts of surfactants and lipid. In this experiment, commercially available polymers were used as proxi (i.e. a measurement of one physical quantity (polymer) as an indicator of the value of another (biofilm)) to model the extracellular matrix of biofilm. The commercially available polymers (also referred to as gels in the following) used in the study were sodium alginate, pectin and hydroxyethyl-cellulose (HEC, Natrosol). Rheological measurements were made on the gels before and after addition of compositions containing the monoglycerides (laurate and/or myristate) in crystalline or amorphous form as well as the bulk cream and aqueous solutions as reference. Brookfield instrument with a T-C spindle was used at a speed of 100 rpm for the measurements.

For each test, following method was used: First, viscosity of the pure gel was measured. Then 10 g of the formulation or composition was added to the gel and the mixture stirred gently. Viscosity was measured immediately before addition of the formulation or composition, immediately after addition of the formulation or composition, 10 min, 1 hour and 24 hours addition of the formulation or composition.

Biofilm Extracellular Matrix Proxi Degradation

For the experiments disclosed herein, viscosity was used to determine the length of the polymers. The longer the polymer, the higher the viscosity. Thus, for the present study, a lowered viscosity of the polymer mixtures was seen as the result of the polymers breaking apart, which in turn provided indications that the given composition was effective in breaking down biofilm.

Three different polymer gels, Natrosol 250HX (Hydroxy-ethylcellulose), Sodium alginate and Pectin, were evaluated and a decrease in the viscosity were detected as the polymers degraded.

As seen in FIG. 1, it was found that the bulk cream efficiently degrades the polymers and causes for decrease in viscosity with time. A decrease in polymer viscosity with time was also be seen in the bulk cream without hydrogen peroxide indicating that other components than hydrogen peroxide could degrade biofilm related polymers of the gels. The decrease in viscosity observed for pure water was a pure dilution effect (no further viscosity decrease is seen with time after the initial mixing).

As further seen in FIG. 2, compositions of crystalline monolaurate and crystalline myristate also were efficient in decreasing the viscosity of the three polymer gels with time, thus, providing a clear indication of polymer degradation independent of hydrogen peroxide. In comparison, a significantly lower decrease in viscosity with time was observed for the composition containing amorphous (non-crystalline) monolaurate. The fact that some decrease was seen for the composition containing amorphous monolaurate was explained by the presence of a small fraction of crystalline material in this formulation or a degradation caused by lactic acid. Indeed, a viscosity decrease was observed over time when lactic acid solution was added to the Natrosol and alginate gels as single active agent. In comparison, no similar decrease in viscosity was seen when water was added to Natrosol or alginate gel. This supported that the lactic acid itself has some effect on degrading biofilm.

Conclusion: Experiments have been performed which show that crystalline monoglycerides can degrade polymer films. Sodium alginate, pectin and hydroxyethylcellulose (HEC, Natrosol) gels were used as models for extracellular polymer substances (EPS) in biofilms. Viscosity measurements on these model gels were done before and after addition of formulations containing either crystalline or amorphous monoglycerides as well as a few reference formulations. It was found that crystalline monolaurate and crystalline myristate efficiently degrades the model gels. It was found that amorphous laurate did not degrade the polymers to the same extend as the crystalline monoglycerides. A slight degradation of Natrosol and Alginate gels could also be seen after addition of a lactic acid solution. No degradation of the polymer gels was found when pure water was added to Natrosol and Alginate gels.

### Example 3. The Effect of Bulk Cream and Components Thereof on Biofilm Produced by Gardnerella vaginalis and Candida albicans

The formulations and compositions used to test the effect of bulk cream and its components on biofilm produced by G. vaginalis and C. albicans were (See Example 1 for the manufacture of bulk cream):
Bulk cream (containing H₂O₂)
Bulk cream (without H₂O₂)
0.3% hydrogen peroxide solution
Monistat 7 (2% Micronazole, Insight Pharmaceuticals)
Metronidazole gel (0.75% Metronidazole, Perrigo)
Phosphate-buffered saline (PBS, biofilm medium)
Water

Gardnerella vaginalis (ATCC® 14018™) and Candida albicans (ATCC® 90028™) were grown on microtiter plates for 48 h at 37° C.±2° C. before being rinsed with PBS to remove planktonic cells. Thereafter the biofilms of the two microorganisms were exposed to the bulk cream and its components (as well as clinical comparators Metronidazole gel and Monistat 7) for 24 h at 37° C.±2° C. The efficacy of formulations in removing biofilm biomass of G. vaginalis and C. albicans was assessed using a crystal violet staining assay and the efficacy of formulations to decrease biofilm viability of G. vaginalis and C. albicans was assessed by plate count method.

Biofilm Biomass Removal

The results of the biomass removal assay are shown in FIGS. 3 and Table 5. As can be seen, bulk cream efficiently removed biofilm mass of both C. albicans and G. vaginalis compared to PBS (biofilm media control). Pure 0.3% H₂O₂ solution and bulk cream without H₂O₂ efficiently diminish the biofilm biomass of G. Vaginalis. Due to high background signal for the remaining formulations it was difficult to draw conclusions of differences between the evaluated formulations. However, it was clear that all tested bulk cream formulations efficiently removed biofilm of C. albicans and G. vaginalis.

TABLE 5
Biofilm biomass of C. albicans and G. vaginalis after
incubation with formulations. Optical density at 570 nm as well
as biofilm forming potential is shown. Mean ± SD is given, n = 3.

| Treatment | C. albicans | G. vaginalis |
|---|---|---|
| t = 0 | 3.42 ± 0.17 High | 0.47 ± 0.135 Medium |
| PBS | 0.88 ± 0.66 High | 0.74 ± 0.08 High |
| Metronidazole gel | n.a* | THTQ** |
| Monistat 7 | 0.33 ± 0.04 Low/no | n.a* |
| Bulk cream | 0.36 ± 0.06 Low/no | 0.29 ± 0.002 Low/no |
| 0.3% H₂O₂ aq. sol. | 0.46 ± 0.11 Medium | 0.27 ± 0.002 Low/no |
| Bulk cream, no H₂O₂ | 0.52 ± 0.11 Medium | 0.34 ± 0.01 Low/no |
| (n = 2)** | | |

**n.a. = not analysed
**THTQ = Too High to Quantify. All three wells of metronidazole gel had residual formulation possibly leading to an inflated value when stained with crystal violet.
*** One well of cream without H₂O₂ had residual formulation and is excluded from the results.

Biofilm Viability

The results of the biomass removal assay are shown in FIG. 4 and Table 6. As can be seen Vernivia bulk cream efficiently removed biofilm mass of both C. albicans and G. vaginalis compared to PBS (Biofilm media control). Due to the antiseptic properties of the peroxide of the composition, the bulk cream is suitable for use against a wide range of biofilms formed by different microorganisms as opposed to antibiotics which are often limited to their target and thus will not target a population of different microorganisms, nor the biofilm. Both pure 0.3% H₂O₂ solution and bulk cream without H₂O₂ efficiently diminished the biofilm biomass of G. vaginalis. Due to high background signal from remaining of formulations, no conclusions of differences between the evaluated formulations were drawn for these. However, it was clear that all tested bulk cream formulations efficiently removed biofilms of C. albicans and G. vaginalis.

TABLE 6
Biofilm viability of C. albicans and G. vaginalis after
incubation with formulations. Log10 CFU/mL. Mean ± SD is given.

| Treatment | C. albicans | G. vaginalis |
|---|---|---|
| t = 0 | 7.72 ± 0.196 | 5.16 ± 0.152 |
| PBS | 7.30 ± 0.173 | 4.77 ± 0.079 |
| Metronidazole gel | n.a* | 2.68 ± 0.144 |
| Monistat 7 | 3.06 ± 0.116 | n.a* |
| Bulk cream | 2.69 ± 0.193 | 2.65 ± 0.321 |
| 0.3% H₂O₂ aq. sol. | 2.90 ± 0.130 | 2.47 ± 0.119 |
| Bulk cream, no H₂O₂ (n = 2)** | 5.48 ± 0.368 | 2.30 ± 0.273 |

**n.a. = not analysed

### Example 4. Effect of Hydrogen Peroxide Concentration on Biofilm

The example is currently under preparation.

Hydrogen peroxide is an important ingredient in Vernivia, acting as an antiseptic agent. Thus, the effect of hydrogen peroxide concentration in the compositions used to treat biofilm is to be tested.

The following vehicles should be used for the experiment (no propellant, i.e. no foam):
Vernivia bulk cream (see Example 1 for composition and manufacture, except hydrogen peroxide concentration as defined below)
Solution of hydrogen peroxide in water 0.3% (control)
Cream containing crystalline monoglycerides but without hydrogen peroxide (control)
Water (placebo)

The specific vehicles were selected in order to be able to separate the effects of monoglycerides and hydrogen peroxide. In addition, only vehicles without propellant were chosen for the study in order to be able to separate the effect of hydrogen peroxide from the effect of the propellant (foaming).

The experiments are expected to show the lowest concentration that degraded any of the tested polymers (see Example 2) and this is considered to be a minimum concentration of hydrogen peroxide for polymer degradation. In this experiment, a hydrogen peroxide concentration of 0.7% to 0.3%, 0.1%, 0.03%, 0.01%, 0.003% and 0.001% in each of the compositions above is to be tested. A concentration of 0.7% corresponded to 200 mM hydrogen peroxide of the final composition while 0.001% corresponded to 0.3 mM of hydrogen peroxide of the final composition. All compositions were started at a concentration of 0.7% hydrogen peroxide. The amount of added formulation should be the same with each test in order not to affect the viscosity of the formulation, which means that for the formulation containing a lower concentration of hydrogen peroxide should be prediluted to make 20 g of added formulation.

The experiment is to establish the minimum concentration of hydrogen peroxide needed for polymer degradation for the each of the compositions tested in this study. The results are expected to indicate that the strongest effect against biofilm is observed for the combinations of hydrogen peroxide and monoglycerides in crystalline form as determined by the viscosity data for the dilutions of respective formulation and supported by laboratory reports from the manufacture and mixing of the compositions. Without wishing to be bound by any particular theory, it is believed that the combination of peroxide and monoglycerides results in a synergistic effect of the mixture. The unique combination of monoglycerides results in an improved disruption of the polymers of the biofilm which in turns result in a lower amount of peroxide needed for causing the antibiofilm (antiseptic) effect.

### Example 5. Inhibitory Effect of Bulk Cream and Components Thereof on Beneficial Bacteria Such as Lactobacillus

The example is currently under preparation.

The vaginal flora contains several species of beneficial bacteria, such as Lactobacillius jensenii, Lactobacillius crispatus, Lactobacillius iners. In this experiment, the effect of Vernivia and components thereof on the growth of lactobacilli is to be investigated. The sustained growth of lactobacilli is of paramount importance with respect to healthy conditions in the vagina. It is therefore important to determine the compatibility of the bulk cream formulation and its components with lactobacilli cultures.

The lactobacilli are grown on agar plates. Once a sustained growth of the bacteria is observed, the bulk cream (see Example 1 for composition and manufacture) is distributed in an even layer on the plates with colonies of lactobacilli. The plates are incubated at 37° C. in the dark for at least 24 hours.

To evaluate the effect of bulk cream on Lactobacilli, the number of colonies and shape of the colonies on the incubated plates should be examined to establish the state, i.e. viability and stress, of the bacteria. The bulk cream formulation is compared to both a negative control, i.e. incubated plates without bulk cream formulation, and to individual components of the bulk cream formulation. The colony number and the morphology can be determined visually and recorded in a laboratory journal.

Preliminary results are expected to indicate that the Lactobacilli were not negatively influenced by the presence of bulk cream formulation. Without the wish to be bound by any particular theory, it is speculated the superior formulation of the bulk cream is ideal for the preservation of beneficial bacteria of the natural flora of humans and animals by its selective effect on microorganisms considered harmful for the host.

What is claimed is:

1. A composition for use in degradation of biofilm or prevention of biofilm formation in a subject, wherein the biofilm is associated with a pathogenic microorganism infection, the composition comprising:
at least one crystalline aliphatic monoglyceride, wherein the at least one crystalline aliphatic monoglyceride is 1-glycerylmonolaurate, 1-glycerylmonomyristate or a combination of both.

2. (canceled)

3. (canceled)

4. The composition of claim 1, wherein the at least one crystalline aliphatic monoglyceride is 1-glycerylmonolaurate and 1-glycerylmonomyristate and optionally the ratio of 1-glycerylmonolaurate and 1-glycerylmonomyristate is from 1 to 10 to 10 to 1.

5. The composition of claim 1, further comprising a peroxide.

6. The composition of claim 5, wherein the concentration of peroxide is less than 0.9% w/w.

7. The composition of claim 6, wherein the peroxide is hydrogen peroxide or benzoyl peroxide.

8. The composition of claim 1, wherein the composition is provided as a mousse, tampon, cream, gel, vaginal suppository or vaginal tablet.

9. The composition of claim 8, wherein the composition further comprises a non-lipophilic propellant when provided as a mousse.

10. The composition of claim 9, wherein the non-lipophilic propellant is air or a gaseous mixture, and wherein the gaseous mixture simulates at least one of air, oxygen, nitrogen, and carbon dioxide.

11. The composition of claim 1, wherein the composition further comprises a solubilizing agent, and wherein the solubilizing agent is selected from polar alcohols, polyethylene glycol, glycerol, propylene glycol, and esters thereof.

12. The composition of claim 1, wherein the pH of the composition is adjusted in accordance with the pH of healthy tissue or a mucous membrane at a site of application.

13. The composition of claim 12, wherein lactic acid is used as a buffer.

14. The composition of claim 13, wherein the lactic acid is the d-isomer of lactic acid.

15. The composition of claim 1, wherein the composition is administered to an infected body cavity or areas of the skin.

16. The composition of claim 15, wherein the infected body cavity or areas of the skin is caused by an infection with a pathogenic microorganism.

17. The composition of 16, wherein the infected body cavity or areas of the skin is caused by an infection with Gardnerella vaginalis, Candida albicans or a combination of both.

18. The composition of claim 15, wherein the infected body cavity or areas of the skin is caused by a lack of commensal microorganisms in the infected body cavity of areas of the skin.

19. (canceled)

20. (canceled)

21. A method for degrading biofilms on a skin surface of the human body, the method comprising:
applying a composition comprising at least one crystalline aliphatic monoglyceride to the skin surface in such a manner that the composition contacts the skin surface, wherein the at least one crystalline aliphatic monoglyceride is 1-glycerylmonolaurate, 1-glycerylmonomyristate or a combination of both.

22. The method of claim 21, wherein the composition further comprises a peroxide.

23. The method of claim 21, wherein the method is a non-therapeutical method.

24. (canceled)

25. (canceled)
